(19) United States
(12) Patent Application Publication
(10) Pub. No.: US 2008/0206292 A1
(43) Pub. Date: Aug. 28, 2008

(54) Cosmetic Method for Targeting of Wrinkles

(75) Inventors: Guillaume Cassin, Villebon sur Yvette (FR); Jean-Thierry Simonnet, Cachan (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'Orel, Paris (FR)

(21) Appl. No.: 11/994,891

(22) PCT Filed: Jul. 4, 2006
(86) PCT No.: PCT/IB06/03359

§ 371 Date: May 2, 2008

(30) Foreign Application Priority Data
Oct. 14, 2005 (FR) 0553122

(51) Int. Cl. A61K 8/02; A61K 31/7076; A61Q 19/08; A61K 9/00

(52) U.S. Cl. 424/401; 514/46; 424/489

(57) ABSTRACT

A subject-matter of the invention is a cosmetic method for caring for wrinkled skin, for the purpose of softening the wrinkles, comprising the successive stages: (a) of topical application to the wrinkled areas of a first composition including a pulverulent phase comprising porous particles which comprise at least one antiwrinkle active principle at least partially absorbed in the said particles, the said composition being substantially devoid of solvent for the said active principle, and (b) of topical application to the skin of the face comprising at least the said areas of a second composition including a fatty and/or aqueous phase comprising a solvent for the said active principle.

A subject-matter of the present invention is a cosmetic method for caring for wrinkled skin, for the purpose of softening the wrinkles, comprising the successive stages: (a) of topical application to the wrinkled areas of a first composition including a pulverulent phase comprising porous particles which comprise at least one antiwrinkle active principle adsorbed on and/or absorbed in the said particles, the said composition being substantially devoid of solvent for the said active principle, and (b) of topical application to the skin of the face comprising at least the said areas of a second composition including a fatty and/or aqueous phase comprising a solvent for the said active principle.

Concern for retaining a youthful appearance of the skin for as long as possible is a preoccupation of most women and also increasingly affects men. Highly varied cosmetic compositions targeted at meeting this need have thus been put onto the market. They are generally care creams or fluids intended to be applied to the whole of the face and neck which comprise antiwrinkle active principles, such as retinol or its esters, ascorbic acid (vitamin C) or various plant extracts. These active principles are also found in some make-up products, such as foundations, which are increasingly tending to offer the consumer not only a good immediate effect with regard to appearance but also a means for lastingly softening skin defects, such as wrinkles, by the biological action of the active principles which they comprise.

While these solutions of the prior art are satisfactory overall, there exists, however, a marked tendency for the products to become technically more complex in order to meet the expectations of consumers with regard to the appropriateness of the products which they use with their type of skin and the associated aesthetic failings, which can vary from one area of skin to another for the same individual.

The Applicant Company has discovered that it is possible to meet this need by providing a cosmetic method with at least two stages preferably employing a compact powder in its first stage and a cream or a gel in its second stage. This method makes it possible to specifically target the wrinkles by carrying antiwrinkle active principles to the heart of the wrinkles while conferring, on the whole face, an overall improvement in its appearance.

The HAPPI database (January 2004), accessible via the Internet, reveals a composition in the form of a pressed powder for the face comprising a dry binder and a pulverulent phase including nylon particles impregnated with hyaluronic acid having a mean particle size of 5 μm (Orgasol 2002 N5 HY COS).

In addition, U.S. Pat. No. 5,876,758 (Examples 17 and 20) reveals anhydrous depigmenting compact powders for the face comprising nylon particles of approximately 20 μm on which magnesium ascorbyl phosphate is adsorbed.

Application EP-1 493 433 also reveals compositions intended for the targeting of active compounds in the pilosebaceous unit for the purpose of treating in particular problems of greasy skin and of hair loss. It is furthermore indicated that the pilosebaceous unit constitutes a potential route of passage for agents of anti-deep-wrinkle type. These compositions include porous particles having a mean diameter by volume of less than or equal to 10 μm which can themselves comprise antiwrinkle active principles, such as vitamin C. These compositions can optionally occur in the form of compact powders, thus comprising binders and a pulverulent phase including the above-mentioned particles.

Finally, microcapsules of 20 to 40 μm available commercially from Cognis under the reference Active Powders and disclosed in Application US 2003/0180369 are known. Thus, the product Active Powders Firmness LS 9689 is composed of such microcapsules carrying a pea extract with antielastase activity and is presented as suitable for incorporation in anhydrous make-up products, such as pressed powders, for the purpose of enhancing the firmness of the skin.

Application WO 98/58628 also reveals anhydrous whitening compositions, optionally in the powder form, which deliver active principles to the stratum corneum, the top layer of the epidermis. These compositions comprise an exfoliating agent, in particular salicylic acid, adsorbed on or in a supporting material, such as a polyamide, having a particle size such that at least 80% of the particles do not exceed 20 μm and preferably of greater than 10 μm. The particles of the supporting material have the role of protecting the exfoliating agent in the event of incompatibility with other active principles of the composition. They include the exfoliating agent, optionally in the form dissolved or dispersed in a hydrophilic supporting medium, such as water, an alcohol or an ether. The application of this whitening composition can be preceded by the application to the skin of a moisturizing base.

However, the use is not suggested in these documents of these compositions of compact powder type for targeting wrinkles in a process comprising the additional stage of application to the skin of a second composition which promotes the release of the antiwrinkle active principles.

Thus, the composition illustrated in the HAPPI database is not intended to be covered by a second composition of cream or gel type but, on the contrary, is intended to be applied above a day cream in order to beautify the complexion. The hyaluronic acid thus deposited on the skin will remain on the surface of the epidermis and, for lack of massaging, cannot penetrate into the wrinkles.

In addition, Application EP-1 493 433 exclusively envisages that the release of active principles adsorbed on and/or absorbed in the particles be carried out by simply bringing the particles into contact with the sebum present in the pilosebaceous follicles. In point of fact, in the case where the compositions of EP-1 493 433 are used to target wrinkles of the face, they are applied to people exhibiting instead (in the case of postmenopausal women in particular) hyposeborrhoea and/or to skin areas generally poor in sebum (in contrast to the ridges of the nose, for example). Inadequate release of the active principle absorbed on the particles will ensue.

Furthermore, the microcapsules with an aqueous core disclosed in Application US 2003/0180369 are capable of releasing the active principle which they comprise without an additional stage. However, in contrast to the porous particles used according to the invention, these microcapsules allow only hydrophilic active principles to be carried. In addition, they include water, which renders them sensitive to evaporation.

Finally, the suggestion is not made either in Application WO 98/58628 to apply a moisturizing base above the composition including the particles impregnated with exfoliant. In point of fact, the Applicant Company has demonstrated that the application of a composition including particles charged with an antiwrinkle active principle, followed by the application of a composition which makes it possible to elease this active principle, makes it possible to target and thus to treat wrinkles more efficiently. The inversion of these stages risks, on the contrary, bringing about either evaporation or penetration into the skin of the moisturizing composition before its aqueous phase mixes with the hydrophilic active principle immobilized on the particles. The particles will thus not be correctly wetted and the active principle will not be released or, at any rate, will be insufficiently released from the particles.

It thus remains desirable to be able to have available cosmetic products which make it possible to target the wrinkles for the purpose of rendering them less obvious, that is to say to carry a greater amount of antiwrinkle active principles into the bottom of the wrinkles than at the surface of the skin, for the purpose of avoiding an economically harmful loss on the unwrinkled surface of the face of the active principles present in the product while allowing homogeneous distribution and good release of the active principle on its site of action.

In point of fact, the Applicant Company has demonstrated that it is possible to achieve this aim by successively applying two given compositions to the skin, the first of which is intended to carry antiwrinkle active principles into the wrinkles and the second of which is intended to make possible the homogeneous distribution and the release of these active principles.

A subject matter of the invention is thus a cosmetic method for caring for wrinkled skin, for the purpose of softening the wrinkles, comprising the successive stages: (a) of topical application to the wrinkled areas of a first composition including a pulverulent phase comprising porous particles which comprise at least one antiwrinkle active principle adsorbed on and/or absorbed in the said particles, the said composition being substantially devoid of solvent for the said active principle, and (b) of topical application to the skin of the face comprising at least the said areas of a second composition including a fatty and/or aqueous phase comprising a solvent for the said active principle.

The application of the first composition makes it possible to visualize the areas of skin treated. That of the second composition makes it possible to homogenize the distribution of the particles charged with active principle, to make them penetrate to the bottom of the wrinkles under the effect of the massaging carried out and to release the vectorized active principle on its site of action. During this stage of the method according to the invention, the powders deposited on the skin in the first stage become invisible under the effect of the wetting by the second composition and the wrinkles may be immediately smoothed out in the case where one and/or other of the compositions include(s) fillers with a soft focus effect. In the latter case, the method according to the invention makes it possible to soften the wrinkles both immediately, by an optical effect, and in the longer term, in a lasting fashion, due to the biological effect of the vectorized active principle.

Another advantage of the present invention is that of making it possible to provide the skin with a large amount of fillers, useful for their effects in optically masking the wrinkles, without discomfort, in comparison with a method comprising the application of a single composition.

Yet another advantage of the present invention is that of making it possible to specifically treat the skin areas requiring specific care while conferring, on the entire skin of the face, a lighter, more matt and more uniform complexion, for example by introducing, into the second composition, active principles and/or fillers which make it possible to obtain these effects. As the stages of the above method are not exclusive, it is also possible to anticipate an intermediate stage between the first and the second stages comprising the application, to the skin, of a composition intended to be applied to the T-area of the face in order specifically to target the pores, such as those disclosed in Application EP-1 493 433, comprising porous particles charged with antiseborrhoeic active principle, for example. It would thus be possible to provide a specific anti-ageing kit for greasy skin.

Within the meaning of the present invention, the term “antiwrinkle active principle” is understood to mean a compound of natural or synthetic origin which produces a biological effect, such as an increase in the synthesis and/or in the activity of certain enzymes, when it is brought into contact with an area of wrinkled skin, this effect having the consequence of reducing the outward show of the wrinkles and/or fine lines.

Examples of antiwrinkle active principles which can be used in the present invention can be chosen from: desquamating agents; antiglycation agents; NO-synthase inhibitors; agents which stimulate the synthesis of dermal or epidermal macromolecules and/or which prevent their decomposition; agents which stimulate the proliferation of fibroblasts and/or keratinocytes; agents which stimulate or reduce the differentiation of keratinocytes; muscle relaxants and/or dermo-decontracting agents; agents for combating free radicals; and their mixtures.

Examples of such additional compounds are: adenosine and its derivatives; retinal and its derivatives, such as retinyl palmitate; ascorbic acid and its derivatives, such as magnesium ascorbyl phosphate and ascorbyl glucoside; tocopherol and its derivatives, such as tocopheryl acetate; nicotinic acid and its precursors, such as nicotinamide; ubiquinone; glutathione and its precursors, such as L-2-oxothiazolidine-4-carboxylic acid; C-glycoside compounds and their derivatives, such as disclosed in particular in Application EP-1 345 919, especially C-β-D-xylopyranoside-2-hydroxypropane, such as disclosed in particular in Application EP-1 345 919; plant extracts and in particular rock samphire extracts and olive leaf extracts, and also plant proteins and their hydro-lysates, such as rice or soybean protein hydrolysates; algal extracts and in particular laminarian extracts; bacterial extracts; sapogenins, such as diosgenin and Dioscorea extracts, in particular wild yam extracts, comprising the latter; α-hydroxy acids; β-hydroxy acids, such as salicylic acid and 5-(n-octanoyl)salicylic acid; oligopeptides and pseudodipeptides and their acylated derivatives, in particular {2-[acetyl(3-(trifluoromethyl)phenyl)amino]-3-methylbutyrylamino}acetic acid and the lipopeptides sold by Sederma under the trade names Matrixyl 500 and Matrixyl 3000; lycopene; manganese and magnesium salts, in particular gluconates; and their mixtures.

Mention may in particular be made, as adenosine derivatives, of non-phosphated adenosine derivatives, such as in particular: 2′-deoxyadenosine; 2′,3′-iso-propylideneadenosine; toyocamycin; 1-methyladenosine; N-6-methyladenosine; adenosine N-oxide; 6-methyl-mercaptopurine riboside; and 6-chloropurine riboside. Other adenosine derivatives comprise adenosine receptor agonists, including phenylisopropyladenosine (“PIA”), 1-methylisoguanosine, N⁶-cyclohexyladenosine (CHA), N⁶-cyclopentyladenosine (CPA), 2-chloro-N⁶-cyclopentyl-adenosine, 2-chloroadenosine, N⁶-phenyladenosine, 2-phenylaminoadenosine, MECA, N⁶-phenethyladenosine, 2-p-(2-carboxyethyl)phenethylamino-5′-N-ethylcarboxamidoadenosine (CGS-21680), (N-ethylcarboxamido)adenosine (NECA), 5′-(N-cyclopropylcarboxamido)adenosine, DPMA (PD 129,944) and metrifudil.

Other adenosine derivatives comprise compounds which increase the intracellular concentration of adenosine, such as erythro-9-(2-hydroxy-3-nonyl)adenine (“EHNA”) and iodotubercidin.

Yet other adenosine derivatives comprise its salts and alkyl esters.

The term “C-glycoside derivative” is understood to mean in particular the compounds disclosed in Application EP-1 345 919, corresponding to the following formula (I):

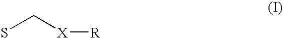

in which:
S represents a monosaccharide or a polysaccharide up to 20 sugar units, in the pyranose and/or furanose form and of the L and/or D series, the said mono- or polysaccharide exhibiting at least one necessarily free hydroxyl functional group and/or optionally one or more optionally protected amine functional groups,
the S—CH₂—X bond represents a bond of C-anomeric nature,
X represents a group chosen from: —CO—, —CH(OH)—, —CH(NR₁R₂)—, —CHR′—, —C(═CHR′)—,
R represents an alkyl, perfluoroalkyl or hydrofluoroalkyl chain which is linear or branched and saturated or unsaturated, a cycloalkyl, cycloperfluoroalkyl or cyclohydrofluoroalkyl ring, comprising from 1 to 18 carbon atoms, or a phenyl or benzyl radical, it being possible for the said chain, the said ring or the said radical optionally to be interrupted by one or more heteroatoms chosen from oxygen, sulphur, nitrogen or silicon and optionally to be substituted by at least one radical chosen from —OR′₁, —SR−₁, —NR′″₁R′₂, —COOR″₂, —CONHR′″₂, —CN, halogen, perfluoroalkyl or hydrofluoroalkyl and/or at least one cycloalkyl, aryl or heterocyclic radical which are optionally substituted,
R′, R₁ and R₂, which are identical or different, have the same definition as that given for R and can also represent a hydrogen and a hydroxyl radical,
R′₁, R′₂, R″₁, R″₂, R′″₁ and R′″₂, which are identical or different, represent a hydrogen atom or a radical chosen from an alkyl, hydroxyl, perfluoroalkyl and/or hydrofluoroalkyl radical which is linear or branched and saturated or unsaturated and which comprises from 1 to 30 carbon atoms.

The first composition used according to the invention comprises particles which have a porous nature which allows them to adsorb a sufficient amount of active principle. They are preferably spherical or substantially spherical and advantageously have a mean diameter, by volume, of greater than 10 μm and of less than 100 μm, advantageously of between 10 and 50 μm, indeed even ranging from 20 to 50 μm.

The mean size by volume of the particles can be measured by various techniques. Mention may in particular be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

Preferably, the sizes and size distributions of the particles of the compositions according to the invention are measured by static light scattering using a commercial particle sizer of MasterSizer 2000 type from Malvern. The data are processed on the basis of the Mie scattering theory. This theory, exact for isotropic particles, makes it possible to determine, in the case of nonspherical particles, an “effective” particle diameter. This theory is described in particular in the work by Van de Hulst, H. C., “Light Scattering by Small Particles”, Chapters 9 and 10, Wiley, New York, 1957.

The particles of the composition are characterized by their mean “effective” diameter by volume D[4,3], defined in the following way:

$$D [ 4 , 3 ] = \frac{{\sum }_{l} {V}_{i} \cdot {d}_{i}}{{\sum }_{t} {V}_{i}}$$

where Vᵢ represents the volume of the particles with an effective diameter dᵢ. This parameter is described in particular in the technical documentation of the particle sizer.

The measurements are carried out at 25° C. on a dilute dispersion of particles obtained from the composition in the following way: 1) dilution by a factor of 100 with the predominant solvent of the phase to be analysed, 2) homogenization of the solution, 3) standing of the solution for 18 hours, 4) recovery of the off-white homogeneous supernatant.

The “effective” diameter is obtained by taking a refractive index of 1.33 for the water and a mean refractive index for the particles which depends on their chemical nature and which is, for example, 1.53 for nylon and 1.45 for silica. The refractive index of the particles can be determined experimentally by the “contrast cancellation” method. By choosing two completely miscible solvents with refractive indices which are relatively distant from one another (ethanol: 1.36, and phenylethyl alcohol: 1.529), it is possible to prepare mixtures having intermediate refractive indices. The test particles are suspended in these various solvent mixtures and the transparency of these solutions is subsequently evaluated using a Hach 2100P® turbidimeter sold by Hach. The refractive index of the particle is equal to that of the mixture of solvents for which the least turbid solution is obtained, that is to say the solution exhibiting the lowest cloudiness, which corresponds to the minimum difference in refractive index between the particles and the solvent.

The BET (Brunauer-Emmett-Teller) method is a method well known to a person skilled in the art. It is described in particular in the Journal of the American Chemical Society, vol. 60, page 309, February 1938, and corresponds to the international standard ISO 5794/1 (annex D). The specific surface measured according to the BET method corresponds to the total specific surface, that is to say that it includes the surface area formed by the pores.

Mention may be made, as particles which can be used according to the invention, of organic particles, for example composed of polyamide (nylon), and inorganic particles, for example composed of silica. Use may be made, as polyamide particles, for example, of particles of nylon 6, of nylon 6,6, of nylon 12 or of nylon 6,12, in particular particles of nylon 12, having a mean particle diameter of 20 μm, sold in particular by Arkema under the trade name Orgasol 2002 D NAT COS 204. Use may be made, as silica particles, of amorphous silica microspheres of 20 μm sold in particular by Asahi Glass under the trade name Sunsphere H-201.

The particles used according to the invention can, if appropriate, be treated at the surface with at least one hydrophobic treatment agent, in particular a non-fluorinated hydrophobic treatment agent, provided that they remain porous.

This hydrophobic treatment agent can be chosen in particular from:
silicones, such as methicones or dimethicones;
fatty acids, such as stearic acid;
metal soaps, such as aluminium dimyristate or the aluminium salt of hydrogenated tallow glutamate;
amino acids, N-acylated amino acids or their salts;
lecithin, isopropyl titanium triisostearate; and
their mixtures.

The N-acylated amino acids can comprise an acyl group comprising from 8 to 22 carbon atoms, such as, for example, a 2-ethylhexanoyl, caproyl, lauroyl, myristoyl, palmitoyl, stearoyl and cocoyl group. The salts of these compounds can be the aluminium, magnesium, calcium, zirconium, zinc, sodium or potassium salts. The amino acid can, for example, be lysine, glutamic acid or alanine.

The porous particles can be present in the first composition according to the invention in a content ranging from 0.01% to 50% by weight, preferably ranging from 0.1% to 30% by weight and preferentially ranging from 1% to 15% by weight, with respect to the total weight of the composition.

In addition, the antiwrinkle active principle can represent at least 0.5%, preferably at least 1% and more preferentially at least 5% by weight, with respect to the total weight of the particles present in the first composition. In addition, the antiwrinkle active principle can represent at most 50% by weight, with respect to the total weight of the particles present in the first composition.

Preferably, the antiwrinkle active principle is present at least partially within the said particles, for example present in a proportion of at least 50% and preferably of at least 70% by weight, more preferably of at least 90% by weight, within the said particles, with respect to the total weight of the active principle present in the first composition.

The particles including the active principle are capable of being obtained according to an impregnation process comprising the following stages:
the dissolution of the active principle to be encapsulated in a volatile solvent, such as water, acetone, isopropanol, dichloromethane or ethanol, in order to obtain an impregnation solution,
the impregnation of the particles by the said impregnation solution,
the evaporation of the solvent until a dry powder is obtained.

In the above process, the term “volatile” is understood to mean a compound which is liquid at a temperature of greater than or equal to 20° C. and which has a vapour pressure of greater than 1 mbar at 20° C. The vapour pressure is defined as the pressure at which a liquid and its vapour are in equilibrium.

This process makes it possible to obtain particles which, once introduced into an anhydrous composition, will not be capable or will be only very slightly capable of releasing the active principle which they comprise. In addition, hydration of the active principle, which may result in some cases in a deterioration in the appearance of the powder, for example in a “caking” phenomenon of the latter, will not occur.

The first composition according to the invention can also comprise pigments and/or fillers, in particular when it is in the form of a compact powder.

The term “pigments” should be understood as meaning white or coloured and inorganic or organic particles of any shape which are insoluble in the composition and which are intended to colour it.

Mention may be made, among inorganic pigments, of titanium dioxide, optionally treated at the surface, zirconium or cerium oxides, and also zinc, iron (black, yellow or red) or chromium oxides, manganese violet, ultramarine blue, chromium hydrate and ferric blue, metal powders, such as aluminium powder or copper powder. The pigments can also be chosen from metal oxide nanopigments, such as titanium dioxide, zinc oxide, iron oxide, zirconium oxide, cerium oxide and their mixtures. The term “nanopigments” is understood to mean pigments having a mean particle size ranging from 1 nm to 500 nm and preferably ranging from 10 nm to 100 nm.

Mention may be made, among organic pigments, of carbon black, pigments of D & C type and lakes, in particular lakes based on cochineal carmine of barium, strontium, calcium or aluminium.

The pigments can be present in the composition in a content ranging from 0.1% to 15% by weight, preferably from 0.5% to 12% by weight and preferentially from 1% to 10% by weight, with respect to the total weight of the composition.

The first composition used according to the invention can comprise an additional pulverulent colouring material other than the pigments described above which can in particular be chosen from pearlescent agents.

The term “pearlescent agents” should be understood as meaning iridescent particles of any shape produced in particular by certain shellfish in their shells or else synthesized. The pearlescent agents can be chosen from white pearlescent agents, such as mica covered with titanium oxide or with bismuth oxychloride, coloured pearlescent agents, such as titanium oxide-coated mica covered with iron oxides, titanium oxide-coated mica covered with in particular ferric blue or chromium oxide or titanium oxide-coated mica covered with an organic pigment of the abovementioned type, and also pearlescent agents based on bismuth oxychloride.

The pearlescent agents can be present in the composition in a content ranging from 0.1% to 50% by weight, preferably from 0.1% to 40% by weight and preferentially from 0.1% to 30% by weight, with respect to the total weight of the composition.

The first composition used according to the invention can, in addition or alternatively, comprise fillers other than the porous particles, the pearlescent agents and the pigments described above.

The term “fillers” should be understood as meaning colourless or white and inorganic or organic particles which are insoluble in the medium of the composition, whatever the temperature at which the composition is manufactured, and which do not colour the composition.

The fillers can be of any shape, platelet, spherical, hemispherical or oblong, whatever the crystallographic form (for example sheet, cubic, hexagonal, orthorombic, and the like). Mention may be made of talc, mica, silica, kaolin, poly-β-alanine and polyethylene powders, powders formed of tetrafluoroethylene polymers (Teflon®), lauroyllysine, starch, boron nitride, polymeric hollow microspheres, such as those of poly(vinylidene chloride)/acrylonitrile, for example Expancel® (Nobel Industrie), or of acrylic acid copolymers, silicone resin microbeads (Tospearls® from Toshiba, for example), particles of polyorganosiloxane elastomers, precipitated calcium carbonate, magnesium carbonate, basic magnesium carbonate, hydroxyapatite, barium sulphate, aluminium oxides, polyurethane powders, composite fillers, hollow silica microspheres and glass or ceramic microcapsules.

The fillers chosen from:
silicone resin powders, such as, for example, Silicone Resin Tospearl 145A from GE Silicone, with a mean size of 4.5 μm;
hollow hemispherical silicone particles, such as, for example, NLK 500, NLK 506 and NLK 510 from Takemoto Oil and Fat;
wax powders, such as Paraffin Wax Microease 114S particles from Micropowders with a mean size of 7 μm;
polyethylene powders, in particular comprising at least one ethylene/acrylic acid copolymer and especially composed of ethylene/acrylic acid copolymers, such as, for example, Flobeads EA 209 particles from Sumitomo (with a mean size of 10 μm);
powders formed of crosslinked organopoly-siloxane elastomer which are coated with silicone resin, in particular with silsesquioxane resin, as disclosed, for example, in U.S. Pat. No. 5,538,793. Such elastomer powders are sold under the names KSP-100, KSP-101, KSP-102, KSP-103, KSP-104 and KSP-105 by Shin-Etsu;
composite powders formed of talc/titanium dioxide/alumina/silica, such as, for example, those sold under the name Coverleaf AR-80 by Catalyst & Chemicals;
   are very particularly suitable for the invention.

This composition can also comprise metal soaps derived from organic carboxylic acids having from 8 to 22 carbon atoms, preferably from 12 to 18 carbon atoms, for example zinc stearate, magnesium stearate, lithium stearate, zinc laurate or magnesium myristate.

The fillers can be present in the composition in a content ranging from 0.1% to 95% by weight, preferably from 1% to 85% by weight and preferentially from 1% to 80% by weight, with respect to the total weight of the composition.

When it is present, the fatty phase of the first composition used according to the invention, referred to as binder when this composition is in the form of a compact powder, is a fatty phase at ambient temperature (25° C.) composed of water-insoluble liquid or waxy compounds which do not have surfactant properties within the meaning of the McCutcheon and CTFA dictionaries. This fatty phase can comprise in particular one or more oils and/or waxes commonly used in compact powders.

Mention may be made, among the oils and waxes which can be used, of soybean oil, grape seed oil, sesame oil, maize oil, rapeseed oil, sunflower oil, cottonseed oil, avocado oil, olive oil, castor oil, jojoba oil or groundnut oil; hydrocarbon oils, such as liquid paraffins, squalane, liquid petrolatum or polydecene; fatty esters, such as isopropyl myristate, isopropyl palmitate, butyl stearate, isodecyl stearate, hexyl laurate, isononyl isononanoate, 2-ethylhexyl palmitate, 2-hexyldecyl laurate, 2-octyldecyl palmitate, 2-octyldodecyl myristate, 2-octyldodecyl lactate, di (2-ethylhexyl) succinate, diisostearyl malate, glyceryl triisostearate or diglyceryl triisostearate; silicone oils, such as polymethylsiloxanes, polymethylphenylsiloxanes, polysiloxanes modified by fatty acids, fatty alcohols or polyoxyalkylenes, or fluorinated silicones; perfluorinated oils; oleic acid, linoleic acid or linolenic acid; higher fatty alcohols, such as cetanol or oleyl alcohol.

The fatty phase can be present in a content ranging from 0.1% to 40% by weight, preferably from 0.1% to 20% by weight and preferentially from 0.1% to 10% by weight, with respect to the total weight of the composition.

The first composition can comprise other ingredients (adjuvants) commonly used in cosmetics, such as preservatives, UV screening agents, thickeners, surfactants or fragrances.

Of course, a person skilled in the art will take care to choose the optional adjuvant or adjuvants added to the first composition according to the invention so that the advantageous properties intrinsically attached to the composition in accordance with the invention are not, or not substantially, detrimentally affected by the envisaged addition.

The first composition used according to the invention is substantially devoid of solvent for the active principle.

This expression is understood as meaning that this composition comprises less than 5% by weight, preferably less than 2% by weight and better still less than 0.5% by weight of a compound, liquid at a temperature of between 20 and 37° C., in which the active principle can be dissolved (that is to say, forms a true solution) to a level of at least 0.1% by weight at a temperature of between 20 and 37° C. by simple stirring with a magnetic bar.

In particular in the case where the active principle absorbed on the particles is hydrophilic, it is thus preferable for the first composition used according to the invention to be an anhydrous composition, that is to say a composition comprising less than 5% by weight of water, indeed even less than 2% by weight of water, better still less than 0.5% by weight of water, in particular devoid of water.

The composition is advantageously provided in the form of a compact powder which can be prepared by mixing the ingredients of the pulverulent phase (particles of polyamide, fillers, pearlescent agents and pigments, which are optional) and by then adding the fatty phase with stirring, the mixture subsequently being milled (for example with a powder blender of Baker type), sieved and then poured into a dish and compacted.

The milled and sieved mixture of the pulverulent phase and of the fatty phase is, for example, compacted using a press, in particular by applying a pressure ranging from 0.5 MPa to 10 MPa and preferably ranging from 1 MPa to 5 MPa.

The compact powder can in particular be applied to the skin using a sponge or a powder puff.

In an alternative form, the first composition used according to the invention can be provided in a form similar to modelling clay and can, in this case, comprise up to 40% by weight of binder.

According to yet another possibility, the first composition can be in the form of a cast product, generally obtained by mixing the ingredients under hot conditions until a liquid is obtained, which liquid is subsequently cast (still under hot conditions) in a receptacle. After returning to ambient temperature, the composition, which has set solid, can be applied to the skin using a powder puff, a sponge, a brush or the finger.

The method according to the invention comprises a second stage of topical application to the skin of the face (comprising at least the areas treated during the first stage) of a second composition including a fatty and/or aqueous phase comprising a solvent for the said active principle. This second composition is thus suited to releasing the active principle absorbed in the porous particles described above. The amount of solvent for the active principle depends on the level of active principle present in the second composition and is adjusted so as to release a maximum amount of active principle on contact with the skin, optionally in combination with the water naturally present on the skin when the active principle is hydrophilic.

The nature of the solvent depends on that of the active principle. The solvent can in particular be chosen from water, ethanol, glycols or oils which are physiologically acceptable. The term “physiologically acceptable” is understood to mean that the solvent is compatible with the skin. Preferably, the solvent is cosmetically acceptable, that is to say that it is not capable, at the level at which it is used in the second composition, of causing unacceptable discomfort (tingling, redness, tightness, and the like) to the user liable to dissuade him from using this composition.

The second composition can be provided in all the formulation forms conventionally used for topical application and in particular in the form of dispersions of the lotion or gel type, of emulsions with a liquid or semiliquid consistency of the milk type, obtained by dispersion of a fatty phase in an aqueous phase (O/W) or vice versa (W/O), or of suspensions or emulsions with a soft, semisolid or solid consistency of the cream or gel type, or also of multiple (W/O/W or O/W/O) emulsions, of microemulsions, of vesicular dispersions of ionic and/or nonionic type, or of wax/aqueous phase dispersions. These compositions are prepared according to the standard methods.

According to a preferred embodiment of the invention, the composition is provided in the form of an O/W emulsion. According to another embodiment, it is provided in the form of a W/O emulsion including at least 50% by weight of internal aqueous phase, which can in particular be obtained according to the “phase inversion temperature (PIT)” technique.

This composition can additionally comprise various adjuvants commonly used in the cosmetics field, such as emulsifiers, including glyceryl fatty acid esters, optionally polyoxyethylenated sugar fatty acid esters, polyoxyethylene fatty acid esters and sorbitan fatty acid esters; fillers, in particular fillers with a soft focus effect, such as colloidal dispersions of silica or of silica/alumina composite filler, or also polyamide fibres; preservatives; sequestering agents; colorants; fragrances; and thickening and gelling agents, in particular acrylamide homo- and copolymers, acrylic homo- and copolymers and acrylamidomethylpropanesulphonic acid (AMPS) homo- and copolymers.

It can also comprise antiwrinkle active principles chosen in particular from those listed above and/or active principles having other properties, in particular antibacterial, antiseborrhoeic, soothing, depigmenting or moisturizing properties, without this list being limiting.

The invention is illustrated in more detail by the nonlimiting example now described.

### EXAMPLE 1

### Double-Action Product for Targeting Wrinkles

A kit is prepared which comprises two products intended to be applied successively to the skin having the following composition:

Product 1: Powder Compact

| | |
|---|---|
| Talc from Luzenac | 69% |
| Polyamide 12 particles comprising | 20% |
| 5% by weight of adenosine | |
| Magnesium stearate | 2.5% |
| Polydimethylsiloxane | 7.31% |
| Silicone wax | 0.15% |
| Silicone resin | 0.04% |
| Preservative | 1% |

### Procedure:

The charged polyamide particles are prepared in the following way: a solution is prepared comprising 2 g of adenosine, 100 ml of water, 0.5 g of an ethylene oxide/propylene oxide/ethylene oxide copolymer (128 EO/54 OP/128 EO—Poloxamer 338 from Uniqema) and 5 g of a 1N hydrochloric acid solution. This solution is kept stirred at ambient temperature until the active principle has completely dissolved. 38 g of nylon 12 (Orgasol 2002 D NAT COS 204-Arkema) are subsequently dispersed in this solution, the dispersion is then transferred into a round-bottomed flask and the water is evaporated on a rotary evaporator at 40° C. for 8 hours. After complete evaporation of the solvent, a powder is thus obtained composed of nylon particles, the pores of which are filled with adenosine in the solid form.

The compact powder comprising these charged particles is subsequently prepared in a way conventional to a person skilled in the art according to a process comprising the preparation of the loose bulk powder, the sieving of the powder at 250 μm and the compacting of the powder in a dish.

Product 2: Cream Gel

The composition of this product is indicated in INCI names:

| | |
|---|---|
| Cyclomethicone | 5% |
| Hydrogenated polyisobutene | 5% |
| Xanthan gum | 0.4% |
| Acrylates/C10-30 alkyl acrylate | 0.25% |
| crosspolymer | |
| Preservatives | 1% |
| Water | 80.65% |
| Glycerine | 5% |
| Dimethicone PEG-7 phosphate | 2% |
| Carbomer | 0.4% |
| Sodium hydroxide | 0.3% |

Procedure: Phases A and B are homogenized at ambient temperature and then the emulsion is prepared by introducing phase A into phase B with vigorous stirring.

The kit obtained can be used in the following way: product 1 is applied each morning to the wrinkles of the crow's foot and of the forehead using a sponge and then product 2 is applied over the whole of the face, including the forehead, by massaging with the fingertips.

### EXAMPLE 2

### Evaluation of the Effects of the Application of a Double-Action Product

A test during which the cosmetic quality and the immediate effects of a double-action product as prepared according to Example 1 were evaluated was carried out on 6 female volunteers having wrinkles and fine lines on the outline of the eyes.

The mode of application was as follows:
1) The powder is withdrawn in 4 passes over the compact using the applicator made of foam (sponge) and deposited by smoothing movements over the underside of the eye and the crow's foot while seeking to cover the wrinkles and fine lines and also the rings around the eyes. The operation is repeated at least 4 times, equivalent on average to 0.010 g of product deposited. Application is completed by a final overall smoothing with the powder over all the areas of the outline of the eye defined above. The area of application is delimited visually by virtue of the white film deposited on completion of this operation, facilitating the subsequent application of the serum.
2) The serum is subsequently applied in the proportion of a pump-action spray pressure equivalent to a deposition of 100 μl to the area of the underside of the eye and of the crow's foot where the compact powder was deposited beforehand. Mixing is immediate. Penetration of the product is brought about by smoothing movements until a curbing effect is noticed under the fingers.

The results obtained for each parameter evaluated are as follows:
Wrinkles and fine lines under the eye and crow's foot:
After application of the compact powder, the wrinkles and fine lines are observed to be accentuated, even more visibly if they are deep.
After application of the superimposed serum, smoothing of the wrinkles is observed, even more visibly with regard to deep wrinkles and thin skin, and, in 3 cases out of 6, the difference from the bare skin is very great.
Loose bags:

Just like the wrinkles, the bags are accentuated when the powder is applied; by virtue of the mechanical action of the serum on the wrinkles, the skin is tauter, like a face lift, and loosening is reduced.

Overall, after successive application of a compact powder and of a serum according to the invention, the skin is immediately smoothed and tauter. This effect is particularly visible on pronounced wrinkles and thin skin, which react better to the concealing effect produced by the compact powder and the tautening effect produced by the serum.

1. A cosmetic method for caring for wrinkled skin, for the purpose of softening the wrinkles, comprising the successive stages: (a) of topical application to the wrinkled areas of a first composition including a pulverulent phase comprising porous particles which comprise at least one antiwrinkle active principle adsorbed on and/or absorbed in said particles, said composition being substantially devoid of solvent for said active principle, and (b) of topical application to the skin of the face comprising at least said areas of a second composition comprising a fatty and/or aqueous phase comprising a solvent for said active principle.

2. The method according to claim 1, wherein the antiwrinkle active principle is selected from the group consisting of: desquamating agents; antiglycation agents; NO-synthase inhibitors; agents which stimulate the synthesis of dermal or epidermal macromolecules and/or which prevent their decomposition; agents which stimulate the proliferation of fibroblasts and/or keratinocytes; agents which stimulate or reduce the differentiation of keratinocytes; muscle relaxants and/or dermo-decontracting agents; agents for combating free radicals; and their mixtures.

3. The method according to claim 1, wherein the antiwrinkle active principle is selected from the group consisting of: adenosine and its derivatives; retinol and its derivatives, such as retinyl palmitate; ascorbic acid and its derivatives, such as magnesium ascorbyl phosphate and ascorbyl glucoside; tocopherol and its derivatives, such as tocopheryl acetate; nicotinic acid and its precursors, such as nicotinamide; ubiquinone; glutathione and its precursors, such as L-2-oxothiazolidine-4-carboxylic acid; C-glycoside compounds and their derivatives, especially C-β-D-xylopyranoside-2-hydroxypropane; plant extracts and in particular rock samphire extracts and olive leaf extracts, and also plant proteins and their hydrolysates, such as rice or soybean protein hydrolysates; algal extracts and in particular laminarian extracts; bacterial extracts; sapogenins, such as diosgenin and Dioscorea extracts, in particular wild yam extracts, comprising the latter; α-hydroxy acids; β-hydroxy acids, such as salicylic acid and 5-(n-octanoyl)salicylic acid; oligopeptides and pseudodipeptides and their acylated derivatives, in particular {2-[acetyl(3-(trifluoromethyl)phenyl)amino]-3-methylbutyrylamino}acetic acid; lycopene; manganese and magnesium salts, in particular gluconates; and their mixtures.

4. The method according to claim 1, wherein said particles have a mean diameter, by volume, of between 10 and 50 ρm.

5. The method according to claim 4, wherein said particles have a mean diameter, by volume, ranging from 20 to 50 μm.

6. The method according to claim 1, wherein said particles have a specific surface, measured by the BET method, of greater than 1 m²/g.

7. The method according to claim 1, wherein said particles comprise polyamide.

8. The method according to claim 1, wherein said particles comprise silica.

9. The method according to claim 1, wherein said particles are present in the first composition in an amount ranging from 1 to 15% by weight.

10. The method according to claim 1, wherein the antiwrinkle active principle represents at least 1% by weight, with respect to the total weight of the particles present in the first composition.

11. The method according to claim 1, wherein the particles including the active principle are capable of being obtained according to an impregnation process comprising the following stages:
the dissolution of the active principle to be encapsulated in a volatile solvent, in order to obtain an impregnation solution,
the impregnation of the particles by said impregnation solution,
the evaporation of the solvent until a dry powder is obtained.

12. The method according to claim 1, wherein the first composition is in the form of a compact powder.

13. The method according to claim 1, wherein the first composition is anhydrous.
